(19) United States
(12) Patent Application Publication
(10) Pub. No.: US 2019/0008662 A1
(43) Pub. Date: Jan. 10, 2019

(54) INTEGRATED SKELETAL IMPLANT AND ITS METHOD OF SURGICAL IMPLANTATION

(71) Applicant: Robert LYE, Brookvale (AU)

(72) Inventor: Robert LYE, Brookvale (AU)

(21) Appl. No.: 16/124,872

(22) Filed: Sep. 7, 2018

Related U.S. Application Data
(63) Continuation of application No. 15/313,770, filed on Nov. 23, 2016, now abandoned.

(30) Foreign Application Priority Data
May 23, 2014 (AU) 2014901958
Nov. 28, 2014 (AU) 2014904832
Dec. 16, 2014 (AU) 2014905084

(51) Int. Cl. A61F 2/78

(52) U.S. Cl. CPC A61F 2/78; A61F 2002/7887

(57) ABSTRACT

The implant comprises a body having at least one end. The body is elongate as it is arranged to substantially mimic a portion of a skeletal bone. The embodiment of the implant is designed to be implanted in the leg of a patient, as a partial replacement for the femur bone of a patient. The at least one end which includes a stepped portion is arranged to, in use, prevent migration of the implant into the bone of a patient. At least part of the stepped portion is covered by a physiologically inert substance, such as niobium for example, to reduce the possibility of infection or an immune reaction at the site at which the implant contacts the flesh of the patient's leg.

### CROSS REFERENCE TO RELATED APPLICATIONS

This application is a continuation of U.S. patent application Ser. No. 15/313,770, filed on Nov. 23, 2016, which is a United States National Phase Application of PCT/AU2015/050274 filed May 25, 2015, and claims priority to Australian Patent Application No. 2014901958 filed May 23, 2014, Australia Patent Application No. 2014904832 filed Nov. 28, 2014, and Australia Patent Application No. 2014905084 filed Dec. 16, 2014, all of which are incorporated by reference herein.

### TECHNICAL FIELD

The present invention relates to a device for osseointegration into a patient. Embodiments of the invention find specific, but not exclusive, use in the provision and installation of an osseointegrable component arranged to replace a portion of a missing femur bone in the leg of a patient. However, it will be understood that the invention has broader application.

### BACKGROUND ART

The following discussion of the background art is intended to facilitate an understanding of the present invention only. The discussion is not an acknowledgement or admission that any of the material referred to is or was part of the common general knowledge as at the priority date of the application.

Osseointegration is a technique which provides amputee patients with a prosthetic implant which is integrated with the skeleton of a patient. That is, an implant where there is direct contact between living bone and the surface of a load bearing implant. Osseointegration dramatically enhances bone and joint replacement surgery by providing much stronger and longer lasting implants, which in turn provides greater quality of life for amputees.

In some currently utilised osseointegration implants, a skeletally integrated implant is connected through an opening in the stump of an amputee to an external prosthetic limb. This allows direct contact to the ground, which provides greater stability, more control and minimizes energy exerted.

As there is a direct connection between the implant and the external prosthetic limb, there is no need for a patient to use a so-called “suction” prosthesis. Patients that are unable to wear a suction prosthesis for long periods of time or those confined to a wheel chair may benefit from osseointegration implants. Indeed, bilateral amputees have been able to become mobile through osseointegration.

It is against this background that embodiments of the present invention have been developed.

### SUMMARY OF INVENTION

In a first aspect, the present invention provides an implant arranged for integration into a skeletal bone of a patient, comprising a body having at least one end, the body being arranged to substantially mimic a portion of a skeletal bone and wherein the at least one end includes a stepped portion arranged to, in use, prevent migration of the implant into the bone of the patient.

The width of the stepped portion may be greater than the width of the body.

The at least one end may be arranged such that the at least one end does not substantially protrude from the skeletal bone.

The stepped portion of the at least one end preferably comprises a coupling portion arranged to, in use, receive a coupling part.

The coupling portion may further comprise a locking pin. The coupling portion may be tapered.

The body may include a coating arranged to assist osseointegration of the implant with the skeletal bone. In one embodiment, the coating includes a porous structure arranged to assist osseointegration of the implant with the skeletal bone. The porous structure may be formed from titanium, which may in turn be formed by a plasma deposition process.

The implant may be sized to replace at least a portion of a human femoral bone. The implant may also have a curved shape, arranged to mimic the curve of a human femoral bone.

In one embodiment, the body of the implant further includes at least one projection which extends along a portion of the body, wherein the projection is arranged to, in use, prevent rotation of the implant relative to the skeletal bone. The projection may be at least one spline. The at least one spline may extend longitudinally along the body of the implant.

The implant may have a second end which is tapered.

A portion of the at least one end of the implant may be coated with a physiologically inert substance. The physiologically inert substance may be niobium.

In another aspect, the present invention provides a surgical reaming device arranged for reaming a first space in skeletal bone to receive an implant arranged for integration into a skeletal bone of a patient, wherein the first space is profiled to receive an enlarged portion of the implant so that the enlarged portion does not migrate into the bone.

The reaming device may include a primary shaft that is arranged to follow a second space for receiving a portion of the implant, wherein the second space is profiled to be too small to receive the enlarged portion.

The primary shaft may be arranged to create the second space through a drilling action.

The reaming device may include a tapered portion extending from a portion of the primary shaft, wherein the tapered portion is profiled to create the first space.

The tapered portion may be truncated so as to create a stepped portion at the base of the first space.

The reaming device may include a textured portion of larger diameter than the primary shaft, wherein the textured portion is arranged to grind out the first space.

The reaming device may include a collar at one end of the tapered portion; wherein the collar is arranged to control the depth of the first and second spaces.

The collar may be profiled to be larger than an opening into the first space.

The reaming device may be manually operated.

The reaming device may be operated by a powered device.

The powered device may be electrical or pneumatic.

In yet another aspect, the present invention provides a method of surgically implanting an implant into a skeletal bone of a patient, the method comprising the steps of:

forming a longitudinal cavity in the bone of the patient, the cavity being arranged to, in use, receive the implant;

wherein the cavity comprises at least one end;

wherein the at least one end of the cavity further comprises a stepped portion formed to substantially mimic the shape of the implant; and

implanting the implant into the cavity.

The longitudinal cavity may be formed by the primary shaft of the reaming device.

The stepped portion may be formed by the tapered portion.

In yet another aspect, the present invention provides a method of surgically preparing a skeletal bone of a patient for receiving an implant, the method comprising the step of forming a longitudinal cavity in the bone of the patient, the cavity comprising at least one end, the cavity arranged to, in use, receive the implant, wherein the at least one end of the cavity further comprises a stepped portion formed to substantially mimic the shape of the implant.

The method may include the step of, prior to forming a longitudinal cavity, inserting an end of the skeletal bone for receiving the implant into a clamping device and restraining it with the clamping device.

The bone may be restrained between a first and a second receiving jaw.

The first and second receiving jaws may be arranged to come together and restrain the bone in a compression clamping arrangement.

The first and second restraining jaws may be located within a central portion of the clamping device.

A removable bucket may be attachable to the clamping device to collect material removed from the skeletal bone.

A front face of the clamping device may be arranged to form a guide for sawing the skeletal bone.

In yet a further aspect, the present invention provides a clamping device for restraining skeletal bone in inserting an implant arranged for integration into a skeletal bone of a patient, including a central portion arranged to receive the skeletal bone and first and second receiving jaws within the central portion arranged to exert a compression clamping force on the skeletal bone.

At least one of the first and second receiving jaws may be movable with respect to the other through a screwing action to exert the compression clamping force.

Handles may be attached to the first and second receiving jaws.

A front face of the clamping device may be arranged to form a guide for sawing the skeletal bone.

The clamping device may include a removable bucket to collect material removed from the skeletal bone.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a side view of an osseointegrative implant in accordance with an embodiment of the present invention;

FIG. 2 is a projected view of an osseointegrative implant in accordance with an embodiment of the present invention;

FIG. 3 is a side view of an osseintegrative implant in accordance with an embodiment of the present invention, when implanted in a femur bone;

FIGS. 4A to 4C are side, perspective and projected views of a first coupling part arranged to couple at one end with the osseointegrative implant in accordance with an embodiment of the invention and at the other end with a prosthetic;

FIG. 5 is a perspective view of a reamer for creating a space in skeletal bone in accordance an embodiment of the present invention;

FIG. 6 is a perspective view of a reamer for creating a space in skeletal bone in accordance an embodiment of the present invention;

FIG. 7 is a side view of a reamer for creating a space in skeletal bone in accordance an embodiment of the present invention when reaming a space in a femur bone;

FIG. 8 is a side view of an osseointegrative implant in accordance with an embodiment of the present invention, when implanted in a femur bone;

FIG. 9 is a side view of a passageway formed in a bone to receive an osseointegrative implant in accordance with an embodiment of the present invention;

FIG. 10 is a perspective view of a clamping device in accordance with an embodiment of the present invention;

FIG. 11 is a perspective view of a clamping device in accordance with an embodiment of the present invention;

FIG. 12 is a perspective view of a clamping device in accordance with an embodiment of the present invention;

FIG. 13 is a perspective view of a clamping device in accordance with an embodiment of the present invention;

FIG. 14 is a perspective view of a clamping device in accordance with an embodiment of the present invention; and

FIG. 15 is an exploded view of a first coupling part arranged to couple at one end with the osseointegrative implant in accordance with an embodiment of the present invention.

### DESCRIPTION OF EMBODIMENTS

Broadly, embodiments of the invention relate to an implant arranged for integration into a skeletal bone of a patient. Such implants are generally referred to as “osseointegrative” implants.

In the ensuing description, like reference numerals in consecutive figures refer to like or functionally identical parts.

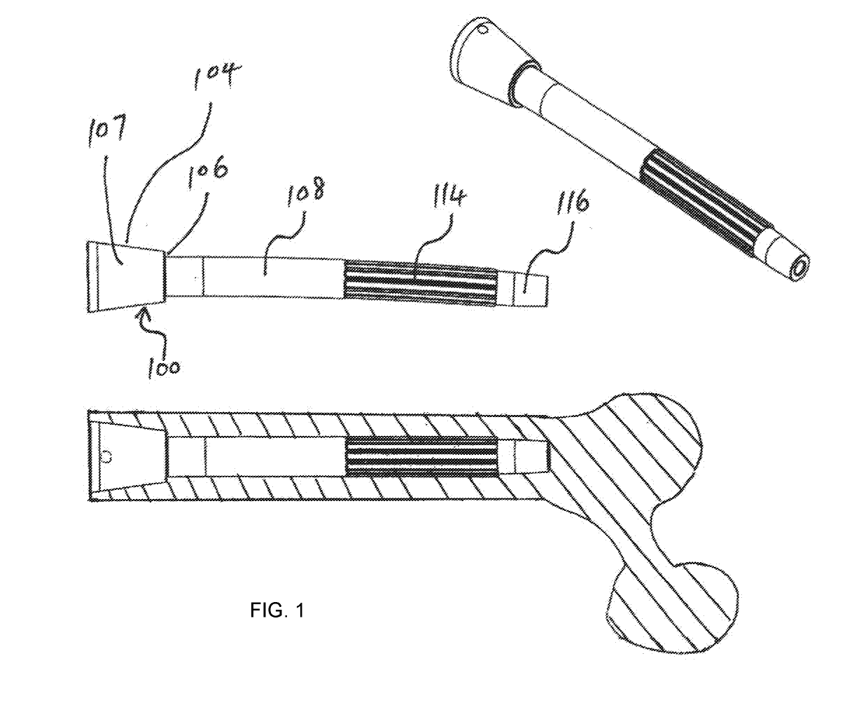

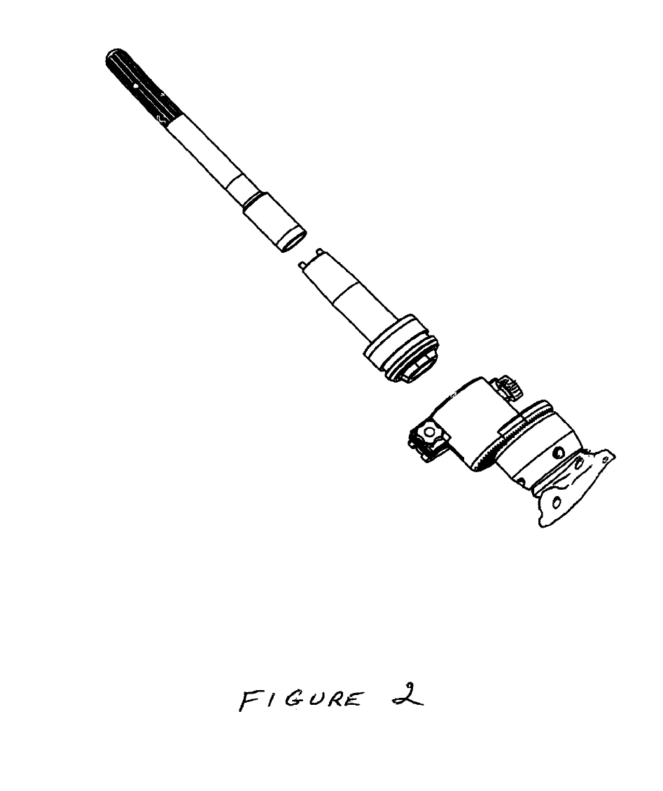

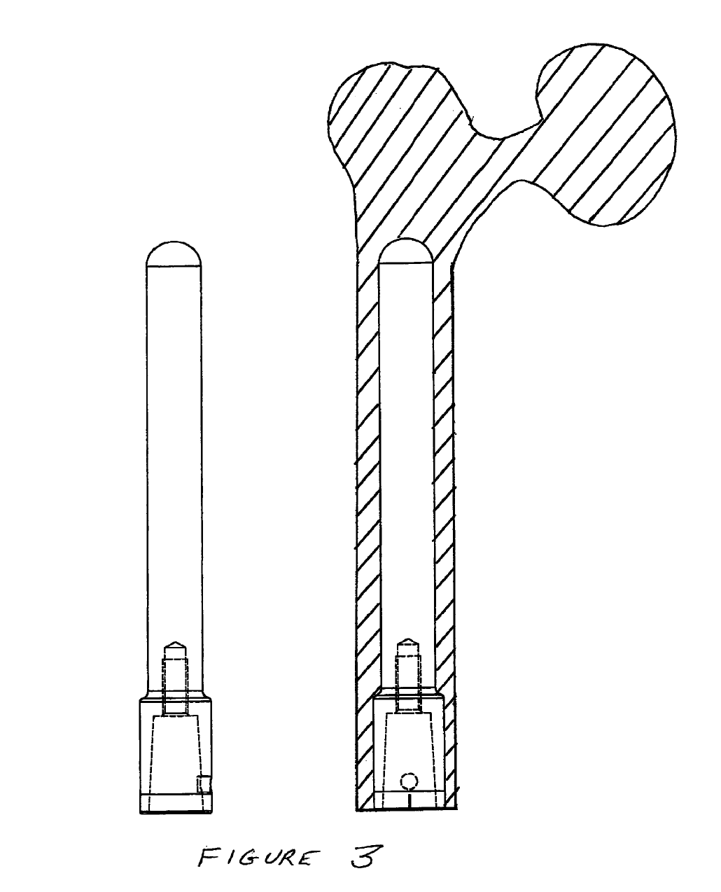

The embodiment described herein, with reference to FIGS. 1 through 3, an implant 100 which comprises a body 102 having at least one end 104. The body 102 is elongate as it is arranged to substantially mimic a portion of a skeletal bone. In the embodiment described herein, the implant 100 is designed to be implanted in the leg of a patient, as a partial replacement for the femur bone of a patient. The patient is an amputee who is seeking to use a prosthetic limb and requires the implant to serve as an “attachment” point for the prosthetic limb.

The at least one end 104 which includes a stepped portion 106 arranged to, in use, prevent migration of the implant into the bone of a patient. Osseointegrative implants suffer from the issue of the ‘end’ of the implant, which is necessarily open to the air and passes through the flesh and skin of a patient, being slowly ‘pushed upwards’ (i.e. upwardly migrating) when the patient wears a prosthetic limb which exerts upward pressure on the implant and therefore can cause the end of the implant to migrate into the bone of the leg of the patient. The embodiment described herein, in contrast, utilizes a stepped portion 106 to prevent such ‘upward migration’ of the implant into the leg of the patient.

At least part of the stepped portion 106 is covered by a physiologically inert substance, to reduce the possibility of infection or an immune reaction at the site at which the implant 100 contacts the flesh of the patients leg. In the embodiment described herein, the physiologically inert substance is niobium, but it will be understood that other coatings may be used, such as gold, or any other coating known or discovered to be physiologically inert. Such variations are within the purview of a person skilled in the art.

The at least one end 104 of the implant 100 further includes a coupling part 107 which is arranged to receive a coupling portion (which will be described in more detail later).

In addition to the stepped portion 106 having a coating, at least a portion of the body 102 may also have a coating (generally denoted by 108), which has the purpose of assisting the implant 100 to integrate into the skeletal bone 110.

In one embodiment, the coating is a suitable porous structure which assists in encouraging bone growth into the porous structure, thereby assisting osseointegration of the implant into the skeletal bone. In one embodiment, the porous structure is formed from titanium which is deposited on the surface of the body 102 by using a plasma deposition process.

The implant has a curved shape which is generally visible at area 112, which is arranged to mimic the curve of a human femoral bone. It will be understood that different types of implants may have different shapes and profiles, as may be required to meet certain skeletal and anatomical constraints. Such variations are within the purview of a person skilled in the art.

The body 102 of the implant 100 further includes at least one projection 114 which extends along a portion of the body 102. The projection is arranged to, in use, prevent rotation of the implant relative to the skeletal bone, by providing ‘grip’ to prevent rotation of the implant 100 when it is located inside the skeletal bone. In the embodiment shown in the Figures, the projection 114 is at least one spline which extends longitudinally along the body of the implant. However, it will be understood that other variations which achieve the same functionality may include the provision of raised patterns (a ‘zig-zag’ pattern), circumferential ridges, or other simple or complex patterns.

The implant 100 also has a second end 116 which is tapered, to allow the patient to also receive an artificial hip implant (or other implant) which can be attached to the leg implant.

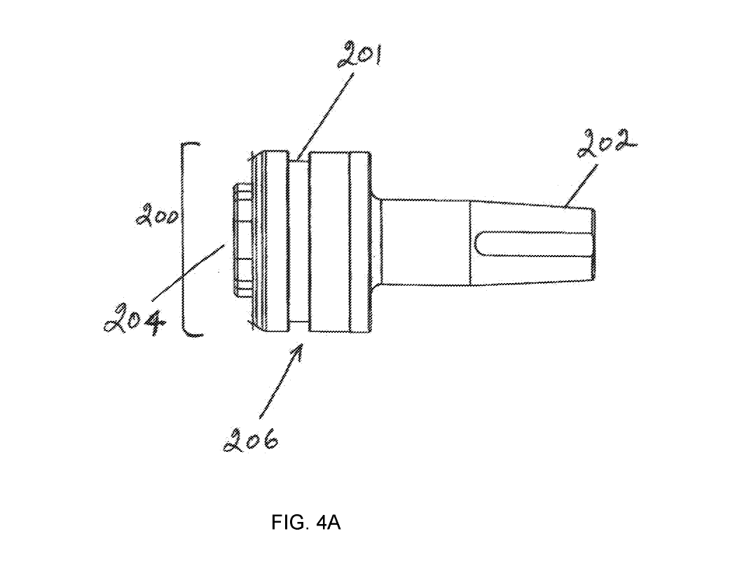

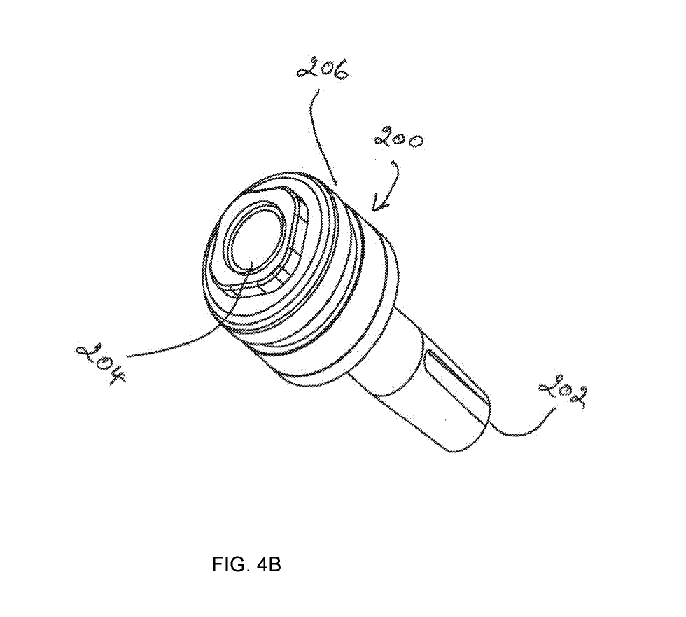

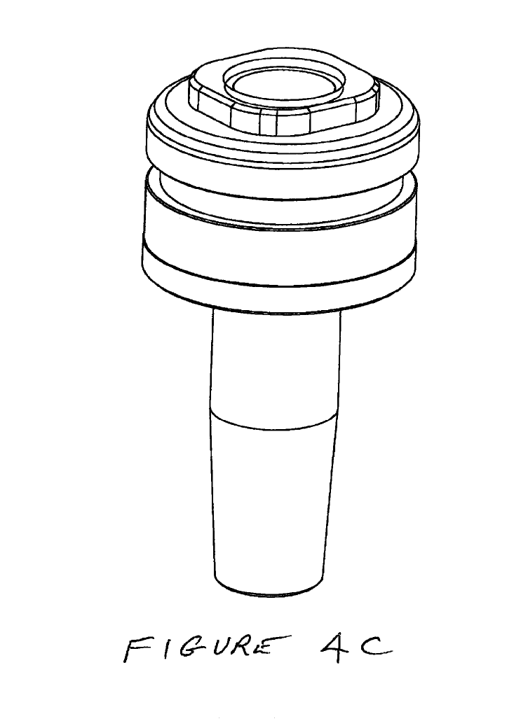

Referring now to FIGS. 4A to 4C there is shown a coupling part which is arranged to cooperate with the implant 100. The coupling part 200 includes a locking slot 202 arranged to lockingly slot into the implant 100. The coupling part also includes a connector engagement boss 204 arranged to connect, either directly or indirectly, with a prosthetic device (not shown), in cooperation with a locking pin channel 206, which is arranged to receive a pin (not shown) to lock the prosthetic (not shown) to the coupling part 200.

Of course, it will be understood that the implant 100 may be manufactured in different sizes, so that the correct size may be provided for different patients of different heights, weights and builds. This may include manufacturing implants of different lengths and/or implants which have different radial profiles. Such variations are encompassed by the broader inventive concept described and defined herein.

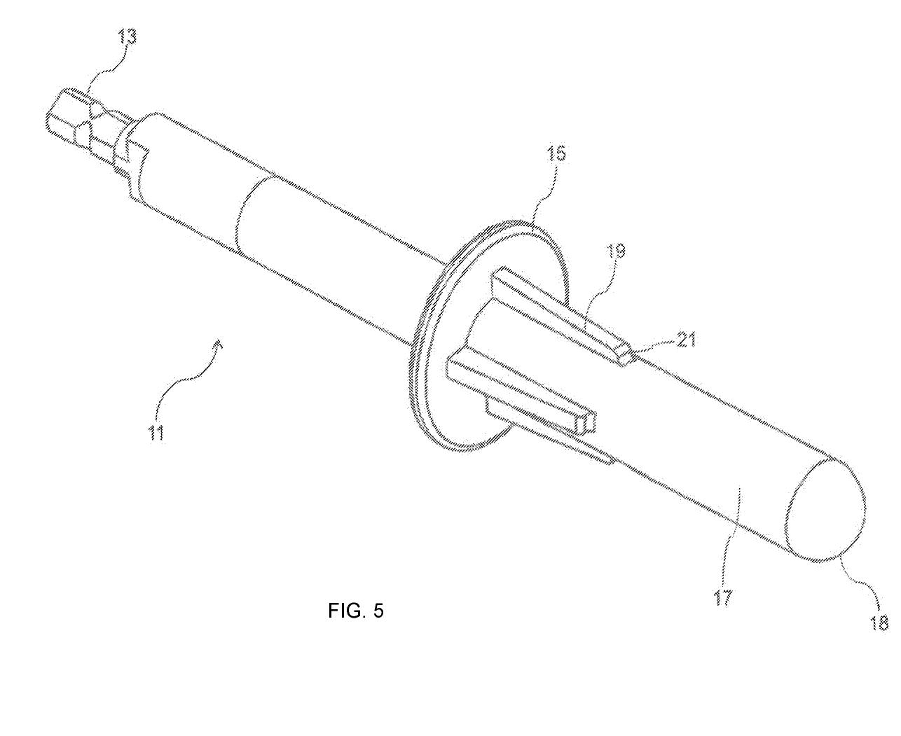

Referring now to FIGS. 5, 6 and 7 there is shown a reaming device/tool 11, 20 for creating a first space in the skeletal bone to receive the stepped portion 106 of the implant 100. The first space is profiled so that the stepped portion 106 rests against bone when inserted in place in the first space so that the stepped portion 106 is restrained by the bone it rests against and does not migrate further into the bone.

In one embodiment a small reamer (not illustrated), longer than reaming tool 11, 20 is used prior to reaming tool 11, 20. The small reamer is used on skeletal bone to drill into the medullary cavity of the bone creating passageway 25.

In an alternative embodiment, the primary shaft 17 of the reaming tool 11 is used to drill into the medullary cavity to form passageway 25 by reaming it out.

With reference to FIG. 5, a reaming tool 11 for creating the first space in accordance with an embodiment of the present invention is shown. The reaming tool 11 includes primary shaft 17 with leading end 18 that is arranged and profiled to be inserted into passageway 25. Attachment coupling 13 is located at an opposite end to leading end 18 and is connectable to a manual or powered tool to rotate the reaming tool 11. Truncated taper portions 19 are arranged as fluting along a portion of the primary shaft 17, distal the leading end 18.

The truncated taper portions 19 include stepped ends 21 to create the truncation of the taper portions 19. When the reaming tool 11 is rotated, either manually or in a powered fashion, and the primary shaft 17 is entered into passageway 25 so that truncated taper portion 19 engages skeletal bone a first space is created that has a stepped base corresponding to stepped ends 21 and tapered sides corresponding to the taper of the truncated taper portion 19. The first space is sized to correspond to the size of the stepped portion 106 of the implant 100.

A collar 15 is located at the end of the truncated taper portions 19 distal to the primary end 18. The collar 15 extends out from the primary shaft 17 in a generally perpendicular fashion. The collar extends perpendicularly outward further than the truncated taper portion 19. This ensures that the collar 15 extends beyond the perimeter of the first space so that the collar cannot enter the first space or into the skeletal bone. Therefore, the collar 15 defines the maximum depth of the first space to be the distance between the stepped ends 21 and the collar 15 as it limits the insertion of the reaming tool 11.

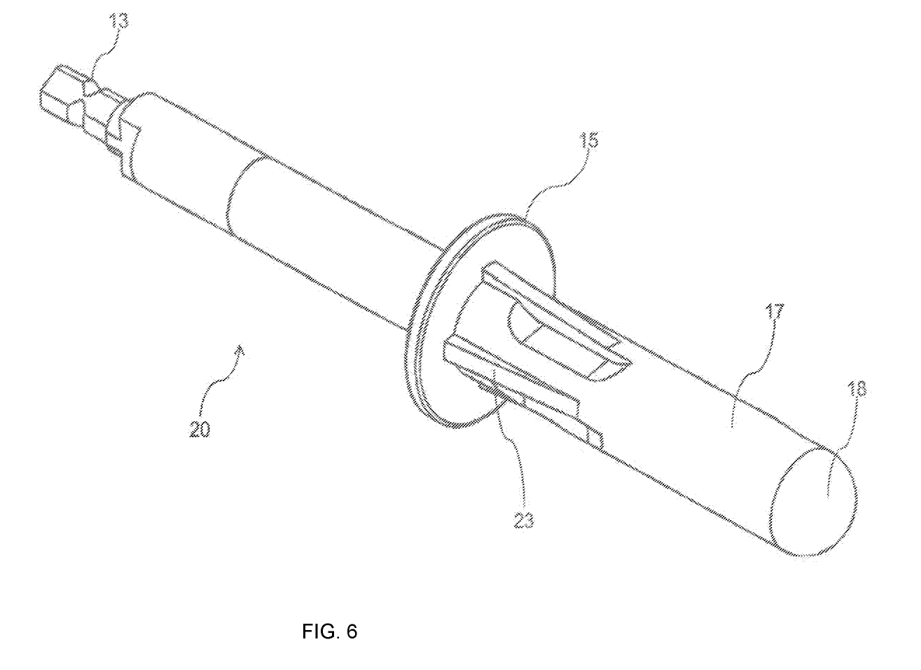

Referring to FIG. 6, an alternative reaming tool 20 for creating the first space in accordance with an embodiment of the present invention is shown. Features of the reaming tool 20 that correspond to those of FIG. 5 have been given the same numbering.

Instead of having truncated taper portions 19, the reaming tool 20 includes tapered portions 23 arranged as fluting that taper all the way onto the surface of the primary shaft 17. When the reaming tool 20 is operated, spinning to perform its reaming function, the tapered portions 23 create a first space in the skeletal bone that is entirely tapered. The stepped portion 106 of the implant 100 is larger at its top than its bottom and therefore is held in the first space so that it cannot migrate into the skeletal bone to which it is attached.

The skilled addressee will recognize that the truncated taper portions 19 or tapered portions 23 could be replaced with alternative reaming structures designed to create a first space larger than passageway 25. For example a grinding block of a larger cross sectional profile than the primary shaft 17, with a textured outer surface could be used to create the first space.

The truncated taper portions 19 or tapered portions 23 could be distinct components to the primary shaft, and could for example be attached to the primary shaft 17 and still fall within the scope of the present invention.

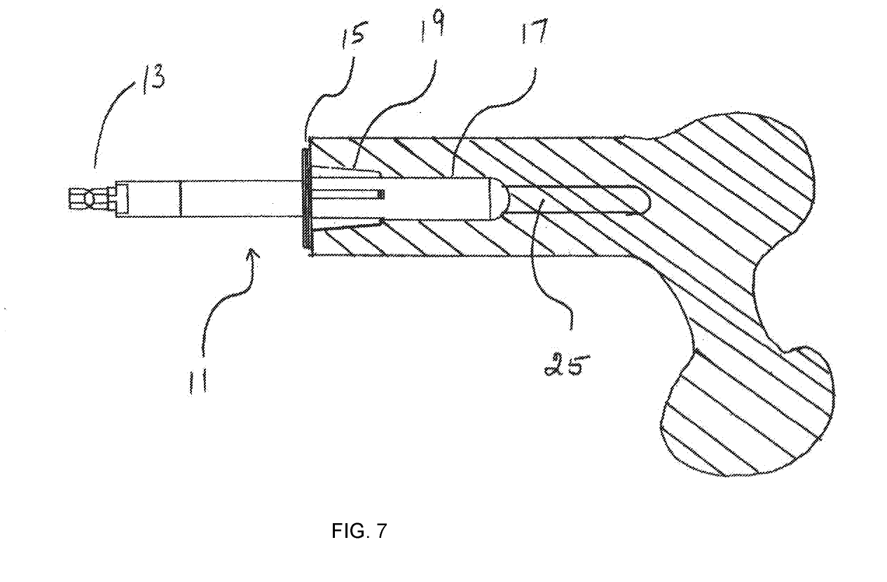

Referring to FIG. 7, the reaming tool 11 is illustrated fully inserted into the skeletal bone.

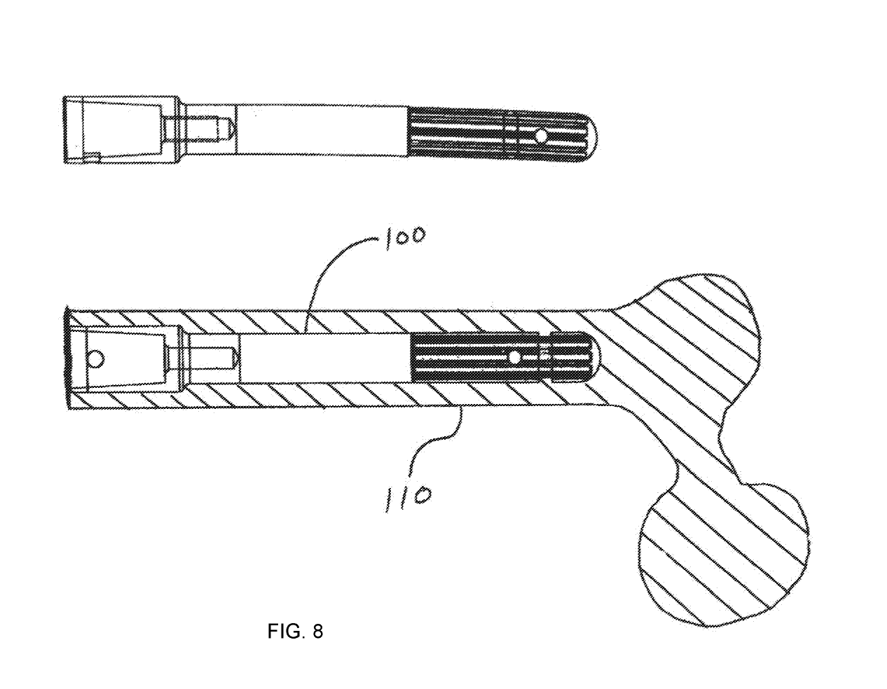

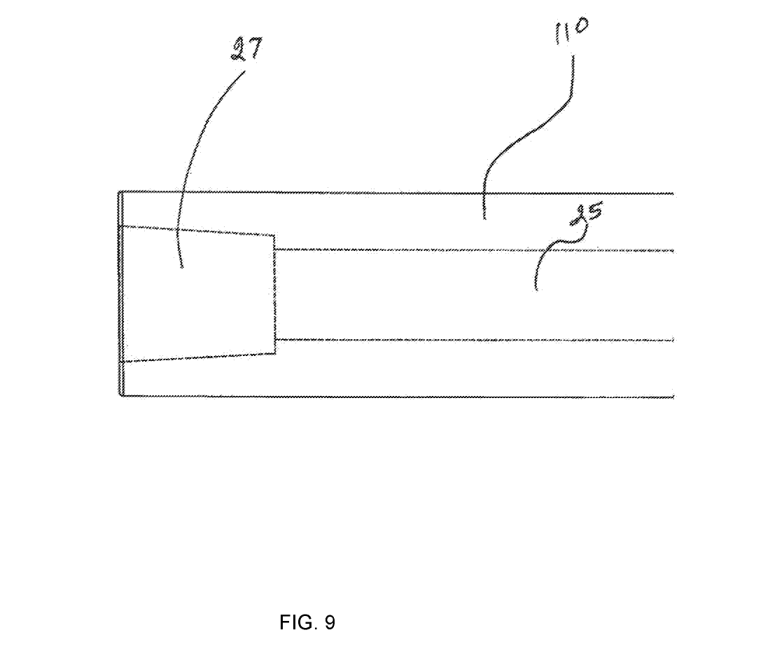

Referring to FIG. 8, the implant 100 is shown inserted in the skeletal bone 110 with the truncated tapered portion inserted into the first space.

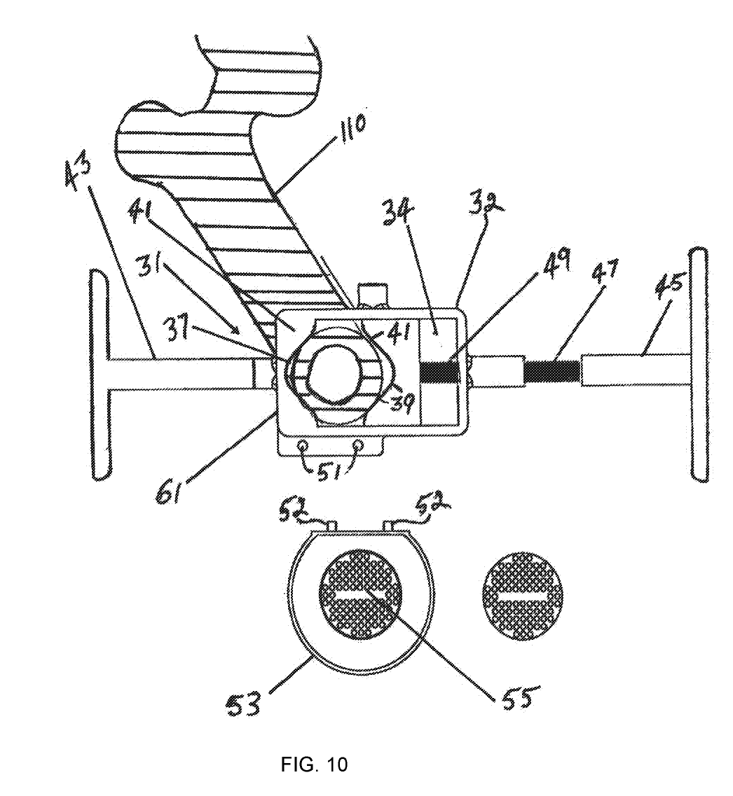

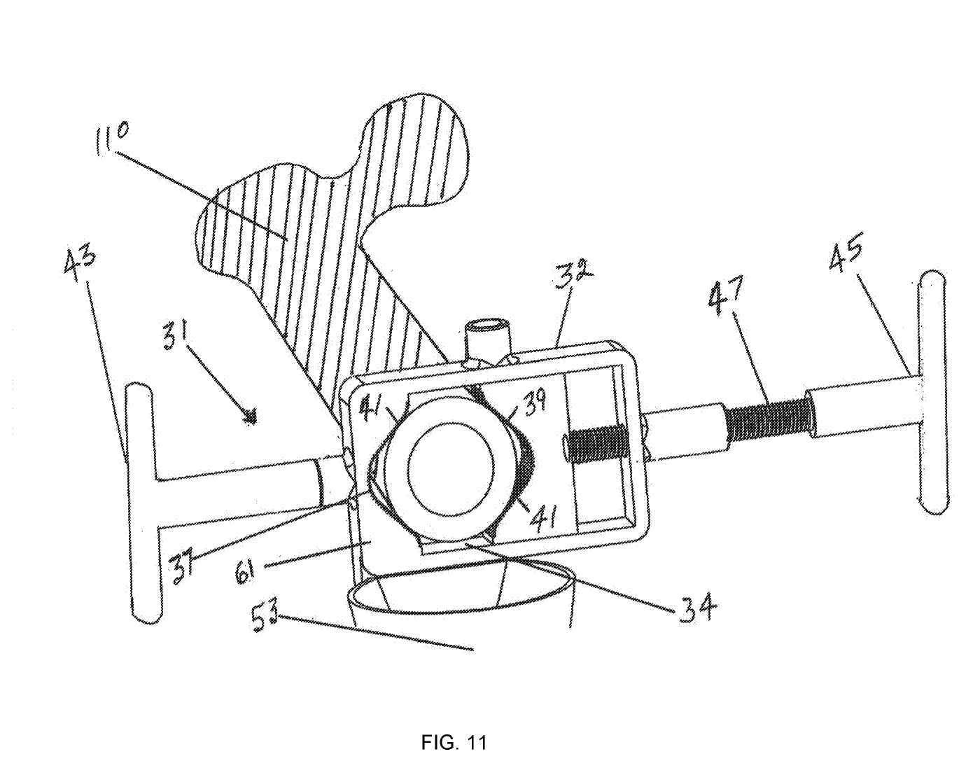

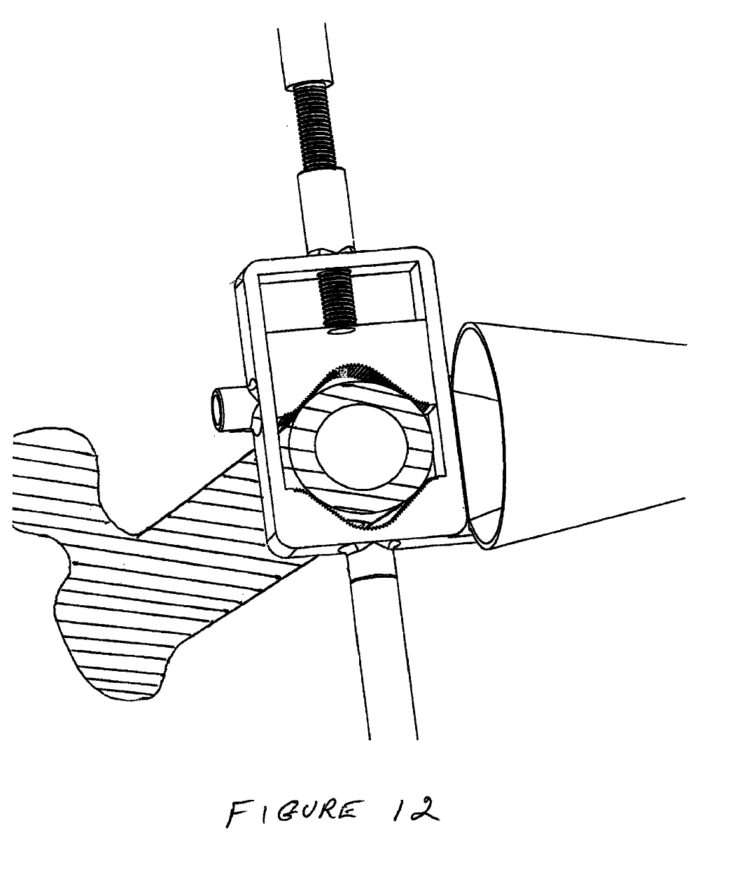

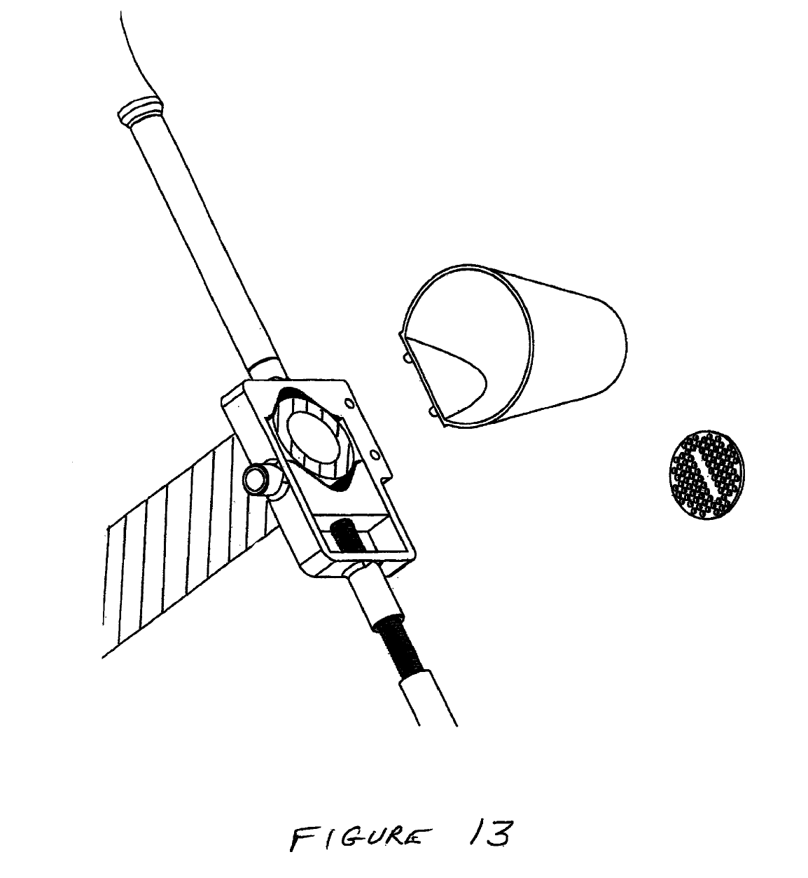

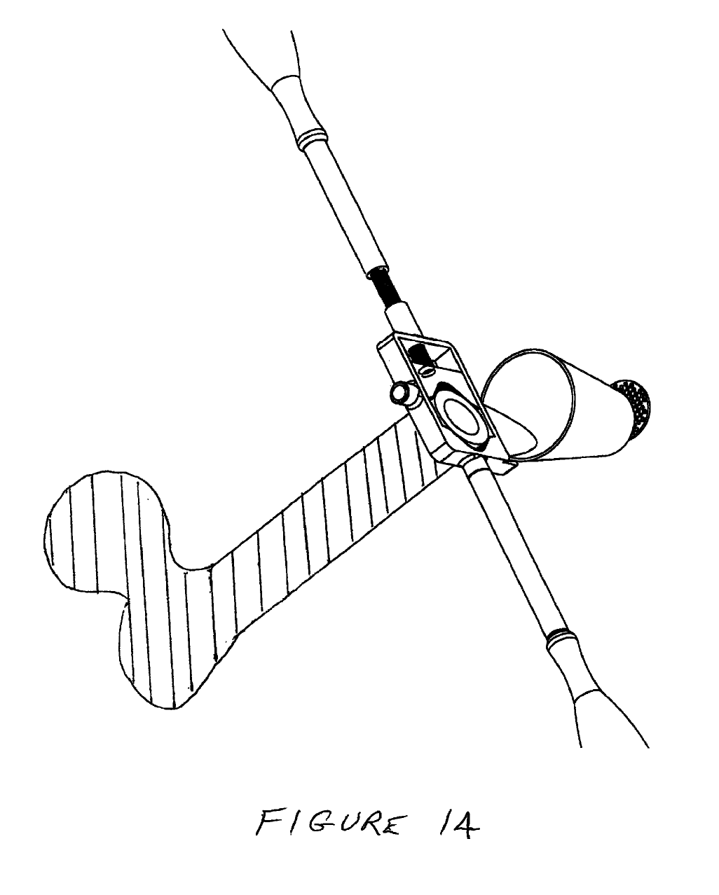

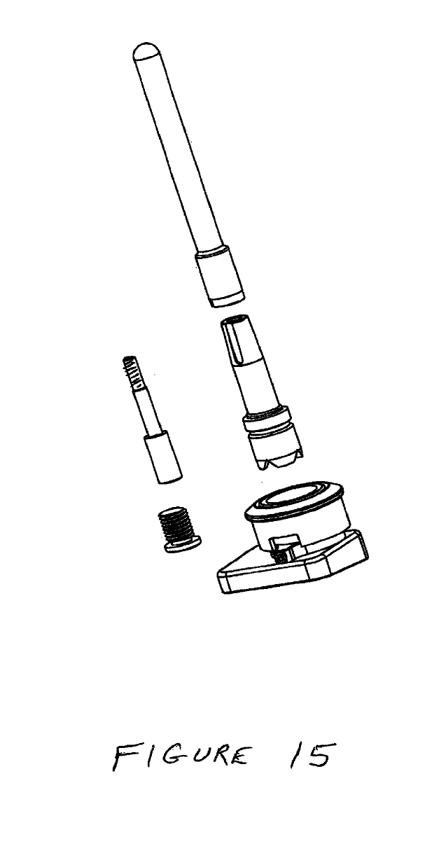

Referring to FIGS. 10 to 14 a clamping device 31 is illustrated for holding the skeletal bone 110 in place when surgery is undertaken for inserting the implant 100 into a patient.

The clamping device 31 includes central portion 32 in which the skeletal bone is to be received and restrained. The central portion 32 includes an internal space 34 that is of a size and shape to allow enlarged portions of a bone, such as the epiphysis regions, or metaphysis or diaphysis regions of different sizes, to pass though the internal space 34. Within the spaced region 34 are a first receiving jaw 37 and a second receiving jaw 39 between which a metaphysis or diaphysis region of the skeletal bone 110 can be held in a compression clamping fashion. Both the first and second receiving jaws 37, 39 have a generally semi-circular profile with teeth 41 along the semi circular profile. The generally semi-circular profile of the first and second receiving jaws 37, 39 allow skeletal bones 110 with different metaphysis or diaphysis diameters to be restrained within the clamping device 31. The front face 61 of the central portion and first and second receiving jaws 37, 39 is flat.

The first receiving jaw 37 is fixed in place within the central portion 32 and internal space 34. Connected to the first receiving jaw 37 is first handle 43. The first handle 43 extends outwards from the central portion 32 so that a user can grip the handle to aid positioning the central portion 32 and first and second receiving jaws 37 around the skeletal bone 110.

The second receiving jaw 39 can be moved within the internal space 34 to increase and decrease the distance between it and the first receiving jaw 37. A second handle 45 is connected to the second receiving jaw 39 and extends outwards from the central portion 32. The second handle 45 includes a male threaded portion 47 that is connected to a first female threaded portion (not shown) in the second receiving jaw 39 and a second female threaded portion 49 in the central portion 32. The threaded engagement between the male threaded portion 47 and the first and second 49 female threaded portions allow the distance between the first and second receiving jaws 37, 39 to be increased or decreased manually through rotation of the second handle 45.

In one embodiment of the present invention both the first receiving jaw 37 and the second receiving jaw 39 can be moved with respect to each other to increase or decrease the distance between them.

In one embodiment of the present invention the second receiving jaw 39, or both the first and second receiving jaws 37, 39 can be moved with respect to each other to increase or decrease the distance between them by using a hydraulic, pneumatic or electric actuator.

To clamp the skeletal bone 110 in place, a metaphysis or diaphysis region of the skeletal bone is placed between the first and second receiving jaws 37, 39 and the two are moved together to clamp the clamping device to the skeletal bone 110 with a compression clamping action.

A bucket 53 is removable attachable to the base of the central portion 32 to receive material that is discharged from the skeletal bone 110. The bucket includes studs 51 that are arranged to be received in recesses 51 in the base of the central portion 32 in a press stud fastening manner. The studs 51 are located on one side of the opening 55 of the bucket so that discharge from the skeletal bone 110 will drop into the bucket.

The skilled addressee will recognize that alternative fastening means can be used to attach the bucket 53 to the central portion 32 such as threaded engagement, clamping engagement or otherwise.

A sieve 53 is insertable into the bucket to capture thicker material such as bone marrow, bone shards or otherwise.

A female threaded portion 57 is located in the top surface of the central portion 32. The threaded female portion can receive an addition handle portion (not shown) to assist in manipulation of the clamping device 31.

### Method of Installing Implant

The following method of installing the implant 100 will be described with respect to a femur. The skilled addressee will readily recognize that the method applies to other skeletal bones of the body where osseointegrative implants can be used and bone depth allows for a first space and smaller passageway 25. The method will also be described with reference to the reaming tool 11. It will be understood that the reaming tool 21 can readily replace the reaming tool 11 for the following method.

The region of the skeletal bone 110 where the implant 100 is to be inserted is exposed. The metaphysis, diaphysis or epiphysis region at the end of the implant region is passed through the central portion 32 of the clamping device 31. The first and second receiving jaws 37, 39 are arranged around the region of the skeletal bone 110 where the implant is to be inserted.

The second receiving jaw 39 is moved closer to the first receiving jaw 37 through rotating the second handle 45. The first and second receiving jaws 37, 39 come together and through teeth 41 exert a compression clamping force on the skeletal bone 110.

With the clamp 31 in place, the portion of skeletal bone 110 extending beyond the clamp 31 is sawn off. The flat front face 61 of the central portion 32 and first and second receiving jaws 37, 39 provide a guide for the sawing that results in an accurate, straight cut through the bone.

The cut off bone falls into the bucket 53. Broaching is performed on the medullary cavity of the skeletal bone 110 to create the passageway 25 of sufficient diameter to receive the body 102 of the implant 110. During the broaching activity, the bone and marrow extracted is collected in the bucket 53.

Alternatively, a small reamer, either mechanical or powered, is drilled into the medullary cavity of the femur along its length to create passageway 25.

The reaming tool 11 has its attachment coupling 13 connected to a manual or powered rotation source. The primary end 18 of the primary shaft 17 is inserted into the created passageway 25 and the stepped ends 21 of the truncated tapered portion 19 are engaged with the end of the femur adjacent the created passageway 25 and operated to ream out bone from the end portion of the femur. The rotation of the truncated tapered portion 19 and pressure applied to the reaming tool 11 reams out bone from the femur. Reaming of bone to form the first space is limited to a depth defined by the collar 15. When the collar 15 abuts the end of the femur to which the reaming tool 11 is applied, the primary shaft 17 and truncated tapered portion 19 can enter the femur no further and the first space has been created, defined by the shape of the truncated tapered portion 19. The reaming tool 11 is then removed from the femur and implant 100 is inserted into the femur so that body 102 is placed into passageway 25.

As the reaming is performed, bone material removed by the creation of the first space is collected in the bucket 53. The bone collected form the broaching and reaming is inserted into the medullary cavity where the implant 100 is inserted to assist with bonding.

Stepped portion 106 of the implant 100 is placed into the first space created by the truncated tapered portion 19. As the stepped portion 106 has a cross section larger than passageway 25, the implant is restrained from migrating into the femur where the stepped portion abuts the base of the first space.

### ADVANTAGES AND INDUSTRIAL APPLICABILITY

One of the advantages of the embodiments and broader invention described herein is that the invention has a stepped portion arranged to stop upward migration of the implant into the bone of the patient.

The implant also preferably includes a porous coating, such as a plasma titanium spray, which acts to induce and assist osseointegration.

Lastly, the embodiment is tapered on the proximal end to allow for future hip implants that may be required by the patient.

### DISCLAIMERS

Throughout this specification, unless the context requires otherwise, the word “comprise” or variations such as “comprises” or “comprising”, will be understood to imply the inclusion of a stated integer or group of integers but not the exclusion of any other integer or group of integers.

Those skilled in the art will appreciate that the invention described herein is susceptible to variations and modifications other than those specifically described. The invention includes all such variation and modifications. The invention also includes all of the features referred to or indicated in the specification, individually or collectively and any and all combinations or any two or more of the steps or features.

Other definitions for selected terms used herein may be found within the detailed description of the invention and apply throughout. Unless otherwise defined, all other scientific, medical, engineering and technical terms used herein have the same meaning as commonly understood to one of ordinary skill in the art to which the invention belongs.

We claim:

1. An implant arranged for integration into a skeletal bone of a patient, comprising a body having at least one end, the body being arranged to substantially mimic a portion of a skeletal bone, wherein the at least one end includes a stepped portion arranged to, in use, prevent migration of the implant into the bone of the patient and wherein the at least one end is configured such that the at least one end does not substantially protrude from the skeletal bone.

2. An implant in accordance with claim 1, wherein the width of the stepped portion is greater than the width of the body.

3. An implant in accordance with claim 1, wherein the stepped portion of the at least one end comprises a first coupling portion arranged to, in use, receive a corresponding coupling part.

4. An implant in accordance with claim 3, wherein the coupling portion further comprises a locking pin.

5. An implant in accordance with claim 3, wherein the coupling portion is tapered.

6. An implant in accordance with claim 1, wherein the body includes a coating arranged to assist osseointegration of the implant with the skeletal bone.

7. An implant in accordance with claim 6, wherein the coating includes a porous structure arranged to assist osseointegration of the implant with the skeletal bone.

8. An implant in accordance with claim 7, wherein the porous structure is formed from titanium.

9. An implant in accordance with claim 8, wherein the porous structure is formed by a plasma deposition process.

10. An implant in accordance with claim 1, wherein the implant is sized to replace at least a portion of a human femoral bone.

11. An implant in accordance with claim 1, wherein a portion of the at least one end is coated with a physiologically inert substance.

12. An implant in accordance with claim 11, wherein the physiologically inert substance is niobium.

13. A method of surgically implanting an implant into a skeletal bone of a patient, the method comprising the steps of:
forming a longitudinal cavity in the bone of the patient, the cavity being arranged to, in use, receive the implant;
wherein the cavity comprises at least one end;
wherein the at least one end of the cavity further comprises a stepped portion formed to substantially mimic the shape of the implant; and
implanting the implant into the cavity.

14. A method of surgically preparing a skeletal bone of a patient for receiving an implant, the method comprising the step of forming a longitudinal cavity in the bone of the patient, the cavity comprising at least one end, the cavity arranged to, in use, receive the implant, wherein the at least one end of the cavity further comprises a stepped portion formed to substantially mimic the shape of the implant.

15. A method in accordance with claim 14, further comprising the step of inserting the implant into the cavity.

16. The method as claimed in claim 14, wherein, prior to forming a longitudinal cavity, an end of the skeletal bone for receiving the implant is inserted into and restrained by a clamping device.

17. The method as claimed in claim 16, wherein the bone is restrained between a first and a second receiving jaw.

18. The method as claimed in claim 17, wherein the first and second receiving jaws are arranged to come together and restrain the bone in a compression clamping arrangement.

19. The method as claimed in claim 18, wherein the first and second restraining jaws are located within a central portion of the clamping device.

20. The method as claimed in claim 16, wherein a front face of the clamping device is arranged to form a guide for sawing the skeletal bone.
